(19) United States
(12) Patent Application Publication
(10) Pub. No.: US 2024/0066249 A1
(43) Pub. Date: Feb. 29, 2024

(54) SYSTEMS AND METHODS FOR DETECTING OCCLUSIONS IN HEADGEAR CONDUITS DURING RESPIRATORY THERAPY

(71) Applicant: ResMed Sensor Technologies Limited, Dublin (IE)

(72) Inventors: Graeme Alexander Lyon, Dublin (IE); Stephen McMahon, Dublin (IE)

(21) Appl. No.: 18/268,379

(22) PCT Filed: Dec. 17, 2021
(86) PCT No.: PCT/IB2021/061975

§ 371 Date: Jun. 20, 2023

Related U.S. Application Data
(60) Provisional application No. 63/130,182, filed on Dec. 23, 2020.

(51) Int. Cl. A61M 16/06; A61M 16/00

(52) U.S. Cl. CPC A61M 16/0672; A61M 16/024; A61M 2205/3334; A61M 2205/3375; A61M 2205/502

(57) ABSTRACT

A system and method detects an occlusion in a respiratory therapy system. The system and method includes determining an acoustic signature for a conduit of a headgear user interface. The determined acoustic signature is analyzed to identify an anomaly in the acoustic signature. In response to the determined acoustic signature being identified as anomalous, a determination is made if the identified anomaly relates to an occlusion of the conduit.

### CROSS REFERENCE TO RELATED APPLICATIONS

The present application claims priority to and the benefit of U.S. Provisional Patent Application No. 63/130,182, filed Dec. 23, 2020, entitled “SYSTEMS AND METHODS FOR DETECTING OCCLUSIONS IN HEADGEAR CONDUITS DURING RESPIRATORY THERAPY,” the disclosure of which is hereby incorporated by reference in its entirety.

### TECHNICAL FIELD

The present disclosure relates generally to systems and methods for detecting occlusions during respiratory therapy, and more particularly, to systems and methods for detecting occlusions in headgear conduits during respiratory therapy.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system. However, some users find such systems to be uncomfortable, difficult to use, expensive, aesthetically unappealing and/or fail to perceive the benefits associated with using the system. As a result, some users will elect not to begin using the respiratory therapy system or discontinue use of the respiratory therapy system absent a demonstration of the severity of their symptoms when respiratory therapy treatment is not used. Occlusion of headgear conduits can lead to incorrect pressure at the user interface without appropriate compensation. Thus, a need exists for systems and method for overcoming user problems with respiratory therapy systems to increase the quality of respiratory therapy including appropriate compensation for headgear conduit occlusions. The present disclosure is directed to solving these and other problems.

### SUMMARY

According to some implementations of the present disclosure, a method for detecting an occlusion within a respiratory therapy system includes determining an acoustic signature for one or more headgear conduits of a headgear user interface during operation of the respiratory therapy system. The determined acoustic signature is analyzed to identify anomalies in the acoustic signature during operation of the respiratory therapy system. In response to the determined acoustic signature being identified as anomalous, a determination is made if an identified anomaly in the determined acoustic signature relates to an occlusion of at least one of the one or more headgear conduits.

According to another implementation of the present disclosure, a method for detecting an occlusion in a respiratory therapy system includes determining an acoustic signature for a conduit of a headgear user interface. The determined acoustic signature is analyzed to identify an anomaly in the acoustic signature. In response to the determined acoustic signature being identified as anomalous, a determination is made if the identified anomaly relates to an occlusion of the conduit.

According to some implementations, a system is disclosed that includes a control system having one or more processors and a memory having stored thereon machine readable instructions. The control system is coupled to the memory, and any of the above aspects can be implemented when the machine executable instructions in the memory are executed by at least one of the one or more processors of the control system.

According to some implementations, a system is disclosed for detecting an occlusion in a respiratory therapy system, the system including a control system having one or more processors configured to implement the method of any one of the above aspects.

According to some implementations, a computer program product is disclosed that includes instructions which, when executed by a computer, cause the computer to carry out the method of any one of the above aspects. According to some aspects, the computer program product is a non-transitory computer readable medium.

The above summary is not intended to represent each implementation or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 is a schematic diagram of air flow through at least a portion a respiratory therapy system including a headgear user interface having conduits, according to some implementations of the present disclosure.

FIG. 4A is a perspective view of an exemplary headgear user interface, according to some implementations of the present disclosure.

FIG. 4B is an exploded view of the exemplary headgear user interface of FIG. 4A, according to some implementations of the present disclosure.

FIG. 5 is another perspective view of at least a portion of the system of FIG. 1, where a user is sleeping with their head to the side with a bed partner, according to some implementations of the present disclosure.

FIG. 6 is a perspective view of an exemplary headgear user interface with one or more occlusions in the headgear conduit, according to some implementations of the present disclosure.

FIGS. 7A and 7B are plots of calculated cepstrums of generated acoustic data for a headgear user interface, according to some implementations of the present disclosure.

FIGS. 8A and 8B are plots of calculated cepstrums of generated acoustic data for another headgear user interface, according to some implementations of the present disclosure.

FIG. 9 is a plot of pressure-flow diagrams for a headgear user interface for a respiratory therapy system, where the solid curved line represents a flow-pressure curve for a headgear user interface with an occluded headgear conduit, and the dashed curved line represents a flow-curve for the headgear user interface with non-occluded headgear conduits, according to some implementations of the present disclosure.

FIG. 10 is a flow diagram of a process for determining if a headgear user interface has an occlusion, according to some implementations of the present disclosure.

FIG. 11 is a flow diagram of a process for determining if a headgear user interface has an occlusion, modifying air flow, and continuing to determine if the occlusion persists, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Various embodiments are described with reference to the attached figures, where like reference numerals are used throughout the figures to designate similar or equivalent elements. The figures are not drawn to scale and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The various embodiments are not limited by the illustrated ordering of acts or events, as some acts may occur in different orders and/or concurrently with other acts or events. Furthermore, not all illustrated acts or events are required to implement a methodology in accordance with the present invention.

Elements and limitations that are disclosed, for example, in the Abstract, Summary, and Detailed Description sections, but not explicitly set forth in the claims, should not be incorporated into the claims, singly, or collectively, by implication, inference, or otherwise. For purposes of the present detailed description, unless specifically disclaimed, the singular includes the plural and vice versa. The word “including” means “including without limitation.” Moreover, words of approximation, such as “about,” “almost,” “substantially,” “approximately,” “generally,” and the like, can be used herein to mean “at,” “near,” or “nearly at,” or “within 3-5% of,” or “within acceptable manufacturing tolerances,” or any logical combination thereof, for example.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as central apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

An RERA event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Therapy for some sleep-related disorders include the application of respiratory therapy systems to treat an individual. A respiratory therapy system includes a user interface that supplies pressurized air to the individual's airway, such as the individuals mouth and/or nose. Headgear user interfaces can include headgear conduits extend along, and are in contact with, a user's face during a therapy session. The headgear conduits may be formed of flexible material that can become compressed, which can lead to occlusion(s) or obstruction(s) in the air flow when the user's head is positioned to the side when lying on the bed, for example, when sleeping on their side, turning their head, or otherwise moving to a position that causes the individual's head to press down on one of the headgear conduits. These occlusions or obstructions of the headgear conduit can disrupt the flow and pressure of supplied air to the system user, and thus, have adverse effects on the respiratory therapy session.

The present technology relates to systems and method for detecting an occlusion within a respiratory therapy system using echo and/or audio data. For example, an acoustic reflection can be used to analyze a headgear user interface that includes one or more conduits where the user interface is coupled to a respiratory therapy device via a tube. The acoustic reflection can be used to detect occlusions in conduit(s) of the user interface. The present technology further contemplates determining a modification to air flow and/or modifying the air flow in response to the identification of an occlusion or obstruction in a headgear conduit to achieve desired pressure settings in the headgear user interface during a respiratory therapy session.

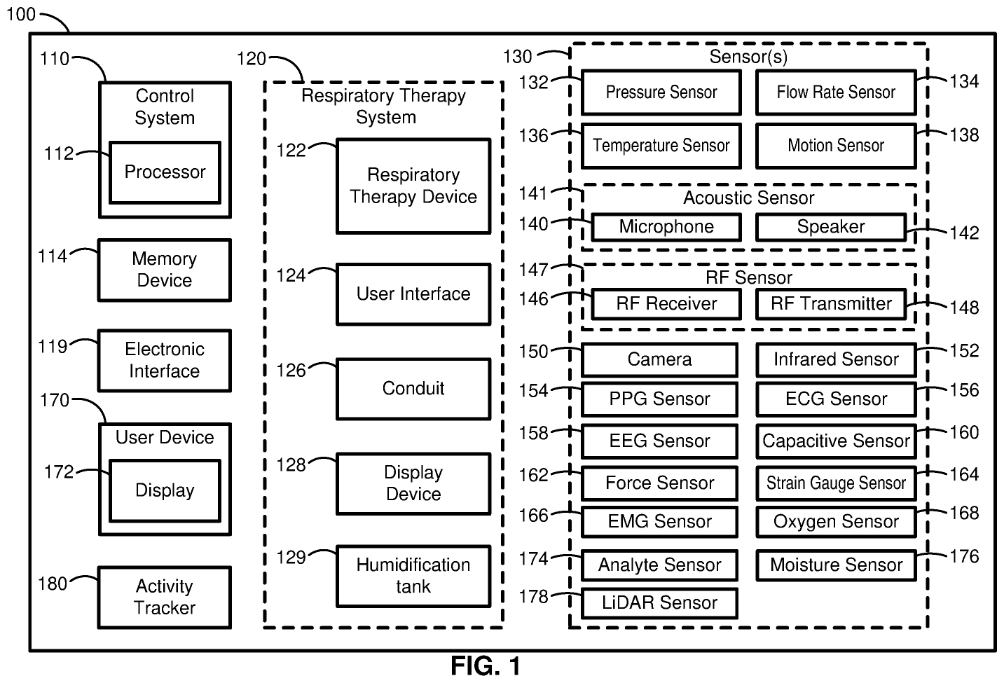

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory therapy system 120, and an activity tracker 180.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 (or any other control system) or a portion of the control system 110, such as the processor 112 (or any other processor(s) or portion(s) of any other control system), can be used to carry out one or more steps of any of the methods described and/or claimed herein. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The medical information can include, for example, data indicative of one or more medical conditions associated with the cohort member, medication usage by the cohort member, or both. The medical information data can further include fall risk assessment associated with the user (e.g., a fall risk score using the Morse fall scale), a multiple sleep latency test (MSLT) test result or score and/or a Pittsburgh Sleep Quality Index (PSQI) score or value. The self-reported feedback can include information indicative of a self-reported subjective sleep score (e.g., poor, average, excellent), a self-reported subjective stress level of the cohort member, a self-reported subjective fatigue level of the cohort member, a self-reported subjective health status of the cohort member, a recent life event experienced by the cohort member, or any combinations thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120. The respiratory therapy system 120 can include a respiratory pressure therapy device 122 (referred to herein as respiratory therapy device 122), a user interface 124 (also referred to as a mask or a patient interface), a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors, such as a blower motor, that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air may also increase the user's oxygen intake during sleep.

As discussed above, the user interface generally is a specific category of user interface, such as direct or indirect (e.g., indirect conduit, indirect frame, etc.). In addition to the specific category, the user interface is generally a specific type of user interface, such as a full face mask, a partial face mask, nasal pillows, etc. In addition to the specific type, the user interface generally is a specific model made by a specific manufacturer. For example, each type of user interface can have different models made by the same manufacturer, with each model varying in shape, size, aesthetic style, manufacturing date, etc.

Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

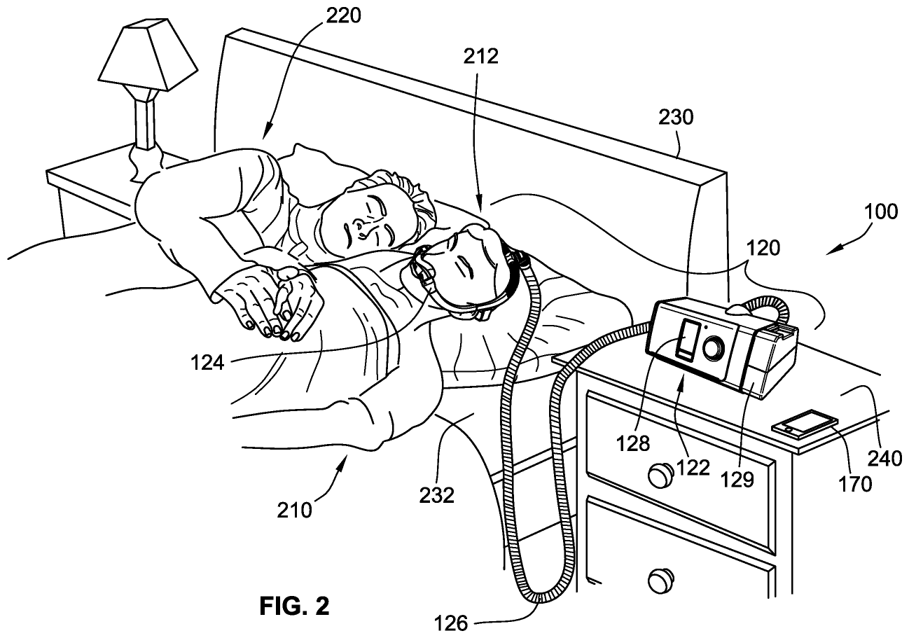

As shown in FIG. 2, in some implementations, the user interface 124 is or includes a facial mask that covers the nose and/or mouth of the user. Alternatively, in some implementations, the user interface 124 can be a nasal mask (see FIG. 2) that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 can include a mouthpiece (e.g., a night guard mouthpiece molded to conform to the user's teeth, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user's airway. Thus, the air pathway generally includes at least a motor of the respiratory therapy device 122, the user interface 124, and the conduit 126.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or a therapy score (such as a myAir™ score, as described for example in WO 2016/061629 and US 2017/0311879, each of which is hereby incorporated by reference herein in their entireties), the current date/time, personal information for the user, questionnaire for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring still to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep stages from physiological data generated by one or more of the sensors, such as sensors 130, are described in, for example, WO 2014/047310, U.S. Pat. Nos. 10,492,720, 10,660,563, US 2020/0337634, WO 2017/132726, WO 2019/122413, US 2021/0150873, WO 2019/122414, US 2020/0383580, each of which is hereby incorporated by reference herein in their entireties. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory therapy device 122, or any combination thereof. The event(s) can include snoring, apneas such as central apneas, obstructive apneas, mixed apneas, and hypopneas, RERAs, a flow limitation (e.g., an event that results in the absence of the increase in flow despite an elevation in negative intrathoracic pressure indicating increased effort), a mask leak (e.g., from the user interface 124), a restless leg, a sleeping disorder, choking, an increased heart rate, labored breathing, an asthma attack, an epileptic episode, a seizure, increased blood pressure, hyperventilation, or any combination thereof. Events can be detected by any means known in the art such as described in, for example, U.S. Pat. Nos. 5,245,995, 6,502,572, WO 2018/050913, WO 2020/104465, each of which is incorporated by reference herein in their entireties.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122, the user interface 124, or the tube 126. The pressure sensor 132 can be used to determine an air pressure in the respiratory therapy device 122, an air pressure in the tube 126, are air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the tube 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user and proximal to the user interface 124, and configured to detect features of the pulse shape, speed, amplitude, or volume. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep-related parameters, which may include one or more events (e.g. respiratory events), as described in further detail herein. The microphone 140 can be coupled to or integrated in the respiratory therapy device 122, the user interface 124, the tube 126, or the user device 170. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the user device 170 (e.g., the microphone 140 is a microphone of a smart phone). The microphone 140 can be integrated into the user interface 124, the conduit 126, the respiratory therapy device 122, or any combination thereof. In general, the microphone 140 can be located at any point within or adjacent to the air pathway of the respiratory therapy system 120, which includes at least the motor of the respiratory therapy device 122, the user interface 124, and the conduit 126. Thus, the air pathway can also be referred to as the acoustic pathway. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are typically audible to a user of the system 100 (e.g., the user 210 of FIG. 2). In one or more implementations, the sound waves can be audible to user of the system 100 or inaudible to the user of the system (e.g., ultrasonic sound waves). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory therapy device 122, the user interface 124, the tube 126, or an external device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. In some implementations, the sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (e.g., a movement, a body position) described in herein such as, for example, a respiration signal, a respiration rate, an inspiration amplitude, an expiration amplitude, an inspiration-expiration ratio, a number of events per hour, a pattern of events, a sleep stage, pressure settings of the respiratory therapy device 122, a mouth leak status, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210 (see FIG. 2). In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the tube 126 or the user interface 124, near the user 210's face, near the connection between the tube 126 and the user interface 124, near the connection between the tube 126 and the respiratory therapy device 122, etc.). Thus, in some implementations, the moisture sensor 176 can be coupled to or integrated in the user interface 124 or in the tube 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

One or more Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the tube 126, the humidification tank 129, the control system 110, the user device 170, the activity tracker 180, or any combination thereof. For example, the microphone 140 and speaker 142 is integrated in and/or coupled to the user device 170 and the pressure sensor 130 and/or flow rate sensor 132 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic event, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home™, Google Nest™ Amazon Echo™, Amazon Echo Show™, Alexa™-enabled devices, etc.). In some implementations, the user device is a wearable device (e.g., a smart watch). The display 172 is generally used to display image(s) including still images, video images, or both. In some implementations, the display 172 acts as a human-machine interface (HMI) that includes a graphic user interface (GUI) configured to display the image(s) and an input interface. The display 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

In some implementations, the system 100 also includes an activity tracker 180. The activity tracker 180 is generally used to aid in generating physiological data associated with the user. The activity tracker 180 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 180 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 180 is coupled (e.g., electronically or physically) to the user device 170.

In some implementations, the activity tracker 180 is a wearable device that can be worn by the user, such as a smartwatch, a wristband, a ring, or a patch. The activity tracker 180 can also be coupled to or integrated a garment or clothing that is worn by the user. Alternatively still, the activity tracker 180 can also be coupled to or integrated in (e.g., within the same housing) the user device 170. More generally, the activity tracker 180 can be communicatively coupled with, or physically integrated in (e.g., within a housing), the control system 110, the memory 114, the respiratory therapy system 120, and/or the user device 170.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc.), or a combination thereof.

While system 100 is shown as including all of the components described above, more or fewer components can be included in a system for implementing the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems for implementing the present disclosure can be formed using any portion or portions of the components shown and described herein and/or in combination with one or more other components.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a headgear user interface with headgear conduits and a nasal mask) can be worn by the user 210 during a sleep session. The user interface 124 is fluidly coupled and/or connected to the respiratory therapy device 122 via the tube 126. In turn, the respiratory therapy device 122 delivers pressurized air to the user 210 via the tube 126 and the user interface 124 to increase the air pressure in the throat of the user 210 to aid in preventing the airway from closing and/or narrowing during sleep. The respiratory therapy device 122 can include the display device 128, which can allow the user to interact with the respiratory therapy device 122. The respiratory therapy device 122 can also include the humidification tank 129, which stores the water used to humidify the pressurized air. The respiratory therapy device 122 can be positioned on a nightstand 240 that is directly adjacent to the bed 230 as shown in FIG. 2, or more generally, on any surface or structure that is generally adjacent to the bed 230 and/or the user 210.

In some implementations, the control system 110, the memory device 114, any of the one or more sensors 130, or a combination thereof can be located on and/or in any surface and/or structure that is generally adjacent to the bed 230 and/or the user 210. For example, in some implementations, at least one of the one or more sensors 130 can be located in one or more components of the respiratory therapy system 120 adjacent to the bed 230 and/or the user 210. The one or more sensors 130 can be coupled to the respiratory therapy system 120, the user interface 124, the tube 126, the display device 128, the humidification tank 129, or a combination thereof.

Alternatively, or additionally, at least one of the one or more sensors 130 can be located in the bed 230 (e.g., the one or more sensors 130 are coupled to and/or integrated in the bed 230). Further, alternatively or additionally, at least one of the one or more sensors 130 can be located at in the mattress 232 that is adjacent to the bed 230 and/or the user 210 (e.g., the one or more sensors 130 are coupled to and/or integrated in the mattress 232). Alternatively, or additionally, at least one of the one or more sensors 130 can be located generally adjacent to the bed 230 and/or the user 210.

Alternatively, or additionally, at least one of the one or more sensors 130 can be located at a fifth position on and/or in the nightstand 240 that is generally adjacent to the bed 230 and/or the user 210. Alternatively, or additionally, at least one of the one or more sensors 130 can be located at a sixth position such that the at least one of the one or more sensors 130 are coupled to and/or positioned on the user 210 (e.g., the one or more sensors 130 are embedded in or coupled to fabric, clothing, and/or a smart device 270 worn by the user 210). More generally, at least one of the one or more sensors 130 can be positioned at any suitable location relative to the user 210 such that the one or more sensors 130 can generate sensor data associated with the user 210.

In some implementations, a primary sensor, such as the microphone 140, is configured to generate acoustic data associated with the user 210 during a sleep session, such as acoustic data having acoustic signatures indicative of occlusion(s) in one or more headgear conduits of a user interface 124 as described herein. For example, one or more microphones (the same as, or similar to, the microphone 140 of FIG. 1) can be integrated in and/or coupled to (i) a circuit board of the respiratory therapy device 122, (ii) the tube 126, (iii) a connector between components of the respiratory therapy system 120, (iv) the user interface 124, (v) a headgear (e.g., straps) associated with the user interface, or (vi) a combination thereof.

Additionally, or alternatively, one or more microphones (the same as, or similar to, the microphone 140 of FIG. 1) can be integrated in and/or coupled to a co-located smart device, such as the user device 170, a TV, a watch (e.g., a mechanical watch or the smart device 270), a pendant, the mattress 232, the bed 230, beddings positioned on the bed 230, the pillow, a speaker (e.g., the speaker 142 of FIG. 1), a radio, a tablet, a waterless humidifier, or a combination thereof.

Additionally, or alternatively, in some implementations, one or more microphones (the same as, or similar to, the microphone 140 of FIG. 1) can be remote from the system 100 (FIG. 1) and/or the user 210 (FIG. 2), so long as there is an air passage allowing acoustic signals to travel to the one or more microphones. For example, the one or more microphones can be in a different room from the room containing the system 100. Based at least in part on an analysis of the acoustic data, a physical obstruction in the airway of a user can be characterize and/or determined.

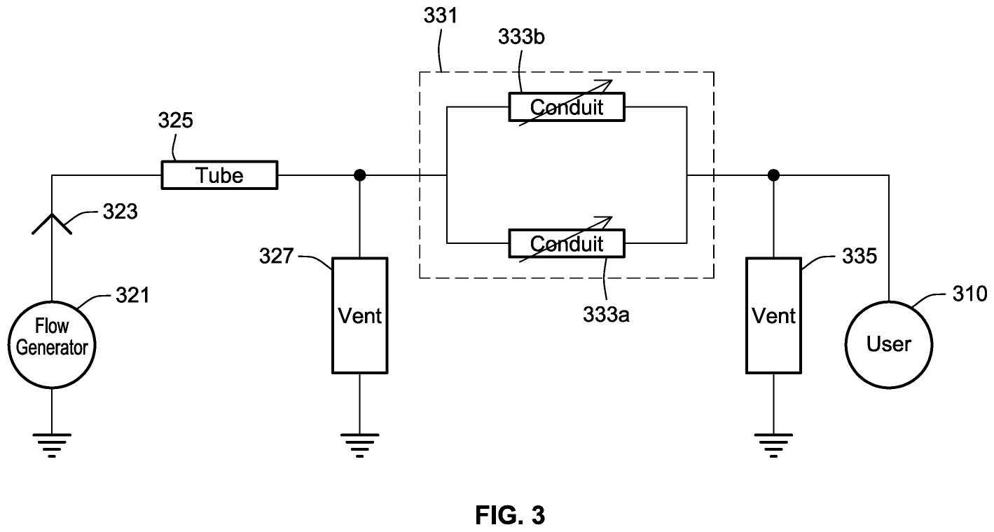

FIG. 3 is a schematic diagram of air flow through at least a portion a respiratory therapy system 100. The schematic of the air flow diagram can also be understood in the context of the respiratory therapy system 120 depicted in FIG. 2. For example, the flow generator 321 is the part of the respiratory therapy system 120 in FIG. 2 that generates an initial pressurized air flow 323, such as through the use of one or more motors that drive one or more compressors in a respiratory therapy device 122. The pressure of the initial pressurized air flow 323 will generally decrease as air flow continues along the air flow pathway due to resistance caused by various pathway components of the respiratory therapy system, such as a tube 325 (e.g., similar to tube 126) and user interface 331 (e.g., similar to user interface 124), along with one or more vents 327, 335. In some implementations, the vents 327, 335 can be positioned before the user interface 331 or by the end of the user interface 331 closest the user 310. In some implementations, the one or more vents 327, 335 are a part of the user interface 331. The user interface 331 then provides the pressurized air to the user 310 at a therapy pressure that is lower than the initial pressure 323 due to the overall air flow resistance along the air flow pathway. For this reason, the flow in the flow generator 321 that determines the initial flow pressure 323 is always set higher than the flow needed to generated the therapy pressure to account for the losses along the air flow pathway.

The present technology is further directed to the user interface 331, and one or more headgear conduits of the user interface 331, such as headgear conduits 333a, 333b, that further impede air flow within the respiratory therapy system 120. For example, the headgear conduits 333a, 333b have a natural resistance during normal operations of the respiratory therapy system and can further become obstructed during a therapy session causing air flow to decrease leading to a drop in therapy pressure. Headgear conduits 333a, 333b, or regions thereof, are typically formed of compressible, resilient material (e.g., elastomer material such as silicone elastomer, optionally comprising nylon/elastane sleeves) and may be subject to compression, and resultant full/partial narrowing of the air passageway within the headgear conduits, when the user's head lies on the headgear conduit such that the headgear conduit is between the user's head and a surface such as a pillow.

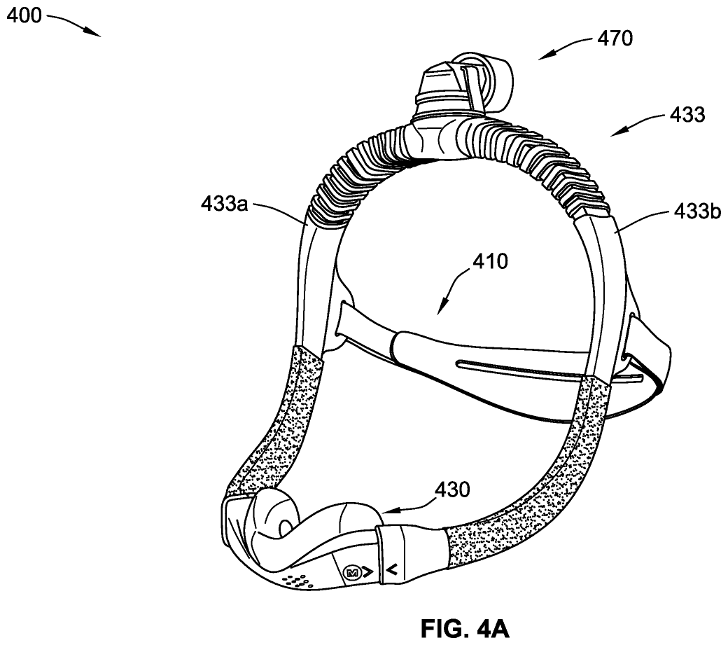

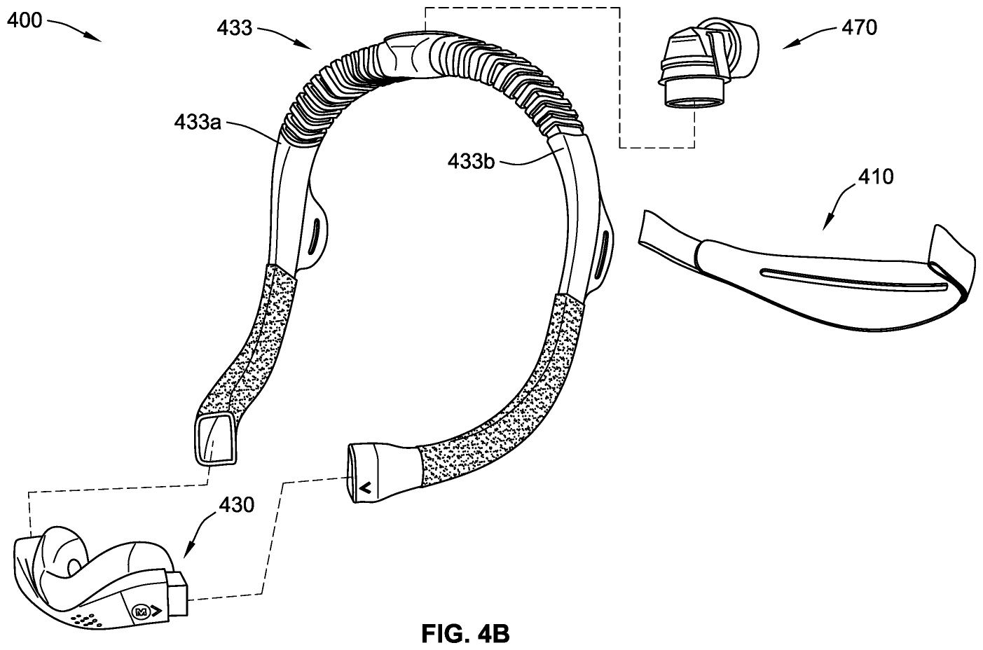

FIGS. 4A and 4B depict a perspective view and an exploded view, respectively, of one implementation of a headgear user interface 400. The headgear user interface 400 includes a cushion 430. The headgear user interface 400 further comprises headgear, that may include a strap 410, a headgear conduit 433, and optionally a connector 470. The headgear components, such as elements 410, 433, 470, are configured to be positioned generally about at least a portion of a user's head when the user wears the user interface 400. The strap 410 can be coupled to the headgear conduit 433 and positioned on the user's head such that the user's head is positioned between the strap 410 and the headgear conduit 433. The cushion 430 is positioned between the user's face and the headgear conduit 433 to form a seal on the user's face. The connector 470 is configured to couple to the headgear, such as to the headgear conduit 433 at one end and a tube (e.g., conduit 126) of the respiratory therapy system at the other end. In other implementations, the connector 470 can be optional and the headgear conduit 433 can alternatively connect directly to tube 126 of the respiratory therapy system.

The headgear conduit 433 may be configured to deliver pressurized air from the tube 126 of the respiratory therapy system to the cushion 430, or more specifically, to the volume of space around the mouth and/or nose of the user and enclosed by the user cushion 430. Thus, the headgear conduit 433 is hollow to provide a passageway for the pressurized air. The head gear conduit 433 is depicted as a combination of two headgear conduits 433a, 433b that are both hollow and provide two passageways for the pressurized air. Alternatively, only one side of the headgear conduit 433 can be hollow to provide a single passageway, or the headgear conduit 433 can include three, four, five, or more headgear conduits. In the implementation illustrated in FIGS. 4A and 4B, the headgear conduits 433a, 433b are configured to be positioned at either side of a user's head/face. The pressurized air can flow from the tube 126 of the respiratory therapy system, through the connector 470 (if present) and the headgear conduit 433, and into the volume of space between the cushion 430 and the user's face. From the volume of space between the cushion 430 and the user's face, the pressurized air reaches the user's airway through the user's mouth, nose, or both.

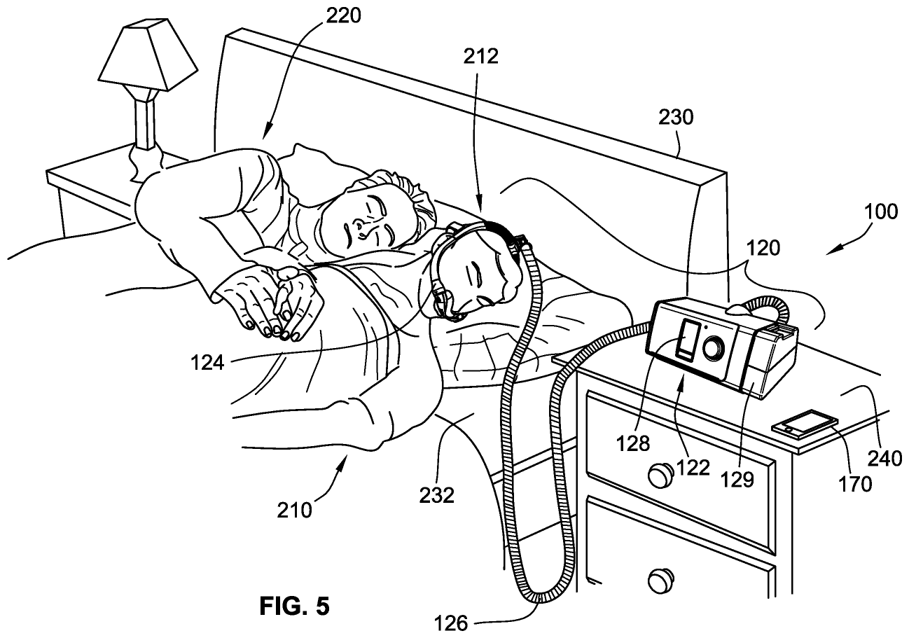

FIG. 5 is another perspective view of at least a portion of the system of FIG. 1 depicting a user sleeping with their head 212 to the side. As in FIG. 2, a user 210 of the respiratory therapy system 120 and a bed partner 220 are located in a bed 230 and are laying on a mattress 232. The user interface 124 (e.g., a headgear user interface with headgear conduits and a nasal mask) can be worn by the user 210 during a sleep session. The user interface 124 is fluidly coupled and/or connected to the respiratory therapy device 122 via the tube 126. In turn, the respiratory therapy device 122 delivers pressurized air to the user 210 via the tube 126 and the user interface 124 to increase the air pressure in the throat of the user 210 to aid in preventing the airway from closing and/or narrowing during sleep.

The respiratory therapy device 122 can be positioned on a nightstand 240 that is directly adjacent to the bed 230 as shown in FIG. 2, or more generally, on any surface or structure that is generally adjacent to the bed 230 and/or the user 210. The head position of the user 210 is depicted such that at least a portion of the side of the user's head 212 is compressing a headgear conduit of the user interface 124, which can cause partial to full occlusions or obstructions in the headgear conduit limiting the air flow within the user interface. Typically, a blocked headgear conduit is highly suggestive that a user is sleeping on their side and may also be an indicator of head and/or body position or positional issues (e.g., a user having apneas only when sleeping on their back) associated with a sleeping disorder. The present technology can therefore be particularly desirable for detecting or understanding a user's positional sleep apnea.

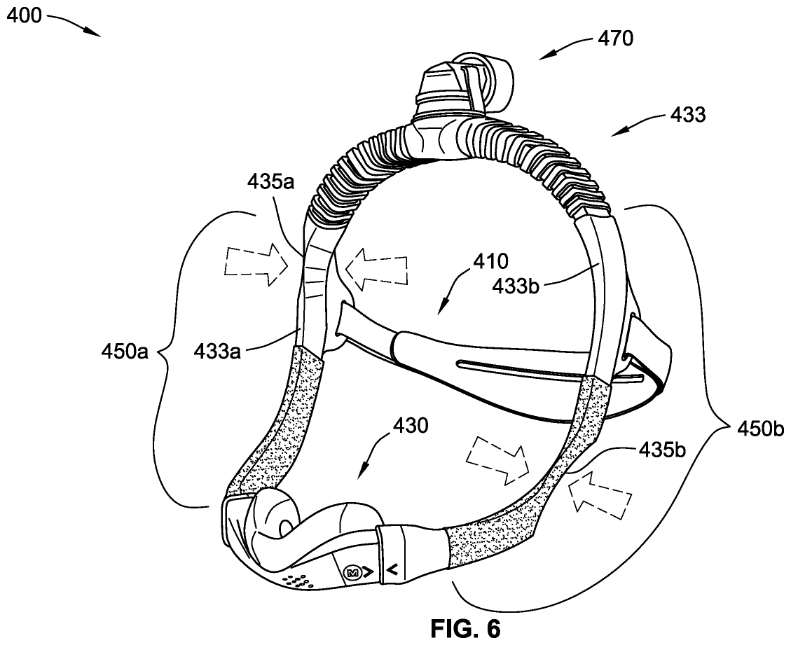

FIG. 6 is a perspective view of the headgear user interface 400 of FIGS. 4A and 4B with occlusions in the headgear conduit 433. As discussed for FIGS. 4A and 4B, the headgear components, such as elements strap 410, headgear conduit 433, and connector 470, are configured to be positioned generally about at least a portion of a user's head when the user wears the headgear user interface 400.

The headgear conduit 433 is configured to deliver pressurized air from the tube 126 of the respiratory therapy system to the cushion 430. As discussed above for FIGS. 4A and 4B, the head gear conduit 433 is depicted as a combination of two headgear conduits 433a, 433b that are both hollow and provide two passageways for the pressurized air. When a user sleeps in a position where their head is on the side, a passageway of the headgear conduit can become compressed leading to one or more occlusions, such as a temple occlusion 435a in headgear conduit 435a and/or a cheek occlusion 435b that at least partially impede the flow of air in the passageway of the headgear conduit 433a or 433b to the cushion 430 causing changes to the therapy session, including the flow of air reaching the user's airway through the user's mouth, nose, or both. Headgear conduits 433a, 433b have specific regions 450a, 450b that can be in particular prone to possible occlusion due to a user sleeping with their head to the side. The length of occlusion regions 450a, 450b for the exemplary user interface 400 is less than the total length of respective headgear conduits 433a, 433b. The occlusion(s) occur at the generally known location of the headgear conduit(s) along the air pathway between the flow generator of the respiratory therapy device and the user's airways.

Obstruction or blockage of a passageway in a headgear conduit, such as headgear conduit 433, can be detected using a microphone that generates acoustic data associated with an acoustic reflection (which may be a plurality of acoustic reflections) of an acoustic signal. For example, the motor of the flow generator of the respiratory therapy device can be the source of such an audio signal where acoustic data is generated associated with acoustic reflections of air flowing through the air pathway of the respiratory therapy system. The microphone can be placed along the air flow path downstream from the motor, for example, between a humidification tank and the exit of the pressurized air from the respiratory therapy device.

Baseline acoustic signatures can be determined from acoustic data for a headgear conduit under normal non-occluded operation (e.g., operation during a therapy session), along with varying degrees of occlusion, and varying locations of occlusion within regions prone for occlusion for a headgear conduit. The baseline acoustic signatures (e.g., cepstrums) can then be compared to actual (operational) acoustic signatures (e.g., cepstrums obtained during a therapy session when a user is wearing a headgear user interface) to identify anomalies in a headgear conduit and determine that the anomaly is due to an occlusion. In some implementations, an estimate of the degree of occlusion in the headgear conduit can be estimated, such as assigning a percentage of blockage, or partial blockage, or full blockage. Baseline data for comparison to operational acoustic signatures can be stored in a digital library of baseline acoustic signatures and/or developed through training algorithms for acoustic data for a particular respiratory therapy system or from a collection of respiratory therapy systems.

In some implementations, it is contemplated that a determined signature can be analyzed by a machine learning algorithm having learned patterns associated with occluded and/or non-occluded headgear conduits during operation of a therapy session, along with learned patterns with varying degrees of occlusion, and varying locations of occlusion within regions prone for occlusion in a headgear conduit.

In some implementations, a method of detecting an occlusion is based on determining if an identified anomaly in a determined acoustic signature is an occlusion by inputting analyzed acoustic data into a machine learning model (which model may have been trained using acoustic data for a headgear conduit obtained under normal non-occluded operation (e.g., operation during a therapy session), along with varying degrees of occlusion, and varying locations of occlusion within regions prone for occlusion for a headgear conduit). In one or more implementations, the machine learning model can be supervised or unsupervised. In one or more implementations, the machine learning model can be a neural network. In one or more implementations, the neural network can be a deep neural network or a shallow neural network. In one or more implementations, the deep neural network can be a convolutional neural network. According to some aspects of the method, the deep neural network includes one or more convolutional layers and one or more max pooling layers.

In some implementations, one or more acoustic signatures can be fed into, for example, a machine learning model (e.g., linear regression, logistic regression, nearest neighbor, decision trees, principal component analysis (PCA), naive Bayes classifier, and k-means clustering, random forest, etc.) and return an identification of an anomaly in a headgear conduit (e.g., occluded, non-occluded, degree of occlusion). In one or more implementations, determining if an identified anomaly in a determined acoustic signature relates to an occlusion of a headgear conduit can be based, at least in part, on one or more determined acoustic signatures matching one or more known signatures of headgear conduits of a headgear interface with a known degree of occlusion (e.g., fully occluded, partially occluded, non-occluded). The determination that an identified anomaly relates to an occlusion can be determined based on a matching determined signatures with known signatures.

In some implementations, it is contemplated that various features or signatures derived from generated acoustic data for an acoustic signal can be determined, such as a peak height or peak heights for a signal over time. Changes between a peak height for a non-occluded headgear conduit and a peak height for an occluded headgear conduit (or varying degrees of occlusion) can be analyzed. The analysis can then identify anomalies in a headgear conduit and determine that the anomaly is due to an occlusion. For example, if a peak height is above a certain threshold value, the analysis would determine that the anomaly is an occlusion in the headgear conduit. If the peak height is below a certain value, the analysis would determine the anomaly is not an occlusion in the headgear conduit.

The present technology contemplates that in response to determining an identified anomaly from a determined acoustic signature relates to an occlusion, information related to a detected occlusion in a headgear conduit is stored for analysis by a body position algorithm module and/or a body movement algorithm module associated with the respiratory therapy system. In some implementations, the information related to a detected occlusion in one or more headgear conduits can be inputted into a body position algorithm module and/or a body movement algorithm module associated with the respiratory therapy system. An occlusion of a headgear conduit would be expected to indicate that a user's head/body is in a side position/orientation.

In some implementations, an acoustic signature in the context of a cepstrum is contemplated to include a certain pattern appearing in the cepstrum. In determining whether an anomaly (e.g., an occlusion) exists in a headgear conduit, the analysis can include looking at one cepstrum or many cepstrums (e.g., combined). A cepstrum can include multiple features that together can be indicative of a blocked headgear conduit. Cepstrum features of interest can include the location, the amplitude, and the shape of various bumps, peaks, and troughs in the cepstrum plot. For a conduit occlusion, a change in the cepstrum in a region on interest of the headgear conduit (e.g., regions 450a, 450b from FIG. 4) is typically characterized by a positive peak in the cepstrum plot corresponding to the point of occlusion (i.e. narrowing) of the headgear conduit. However, the conduit occlusion may additionally or alternatively be characterized by one or more negative peaks in the cepstrum plot corresponding to the points of widening of the acoustic pathway in the vicinity of the point of occlusion of the headgear conduit. Other features of interest can include the peak height (amplitude), the peak prominence, the skewness, the kurtosis, areas above/below lines of intersection, location or amplitude of fiducial points, frequency content, the number of peaks, standard deviation, and the distance between peaks or any combination of the above.

In some implementations, processing or preprocessing can also be completed on the audio data, such as filtering or first or second derivatives or integrals. For example, analysis of generated acoustic data can include calculating a derivative of the cepstrum to determine a rate of change of the cepstrum signal. The derivative of the cepstrum can provide additional information that is used to detect conduit occlusions. The derivative of the cepstrum also allows the identification of alternate features from the same input. For example, the derivative allows easier extractions of features such as the gradient of a portion of, or entire, cepstrum signal which may aid in identifying turning points (zero-crossings in the gradient). The alternate features may for example include mean, maximum, and/or minimum gradient, number of zero crossings, etc.

Although the present disclosure describes the determination of an acoustic signature in the context of a cepstrum for the detection of conduit occlusions, it is contemplated that acoustic signatures may be detected by similar operations in one or more spectrums, time domain, auto-correlation, discrete cosine transform, cross-correlation, Mel-Frequency cepstral coefficients, linear predictive coding, wavelet decomposition, cepstrum, power cepstrum, and/or complex cepstrum.

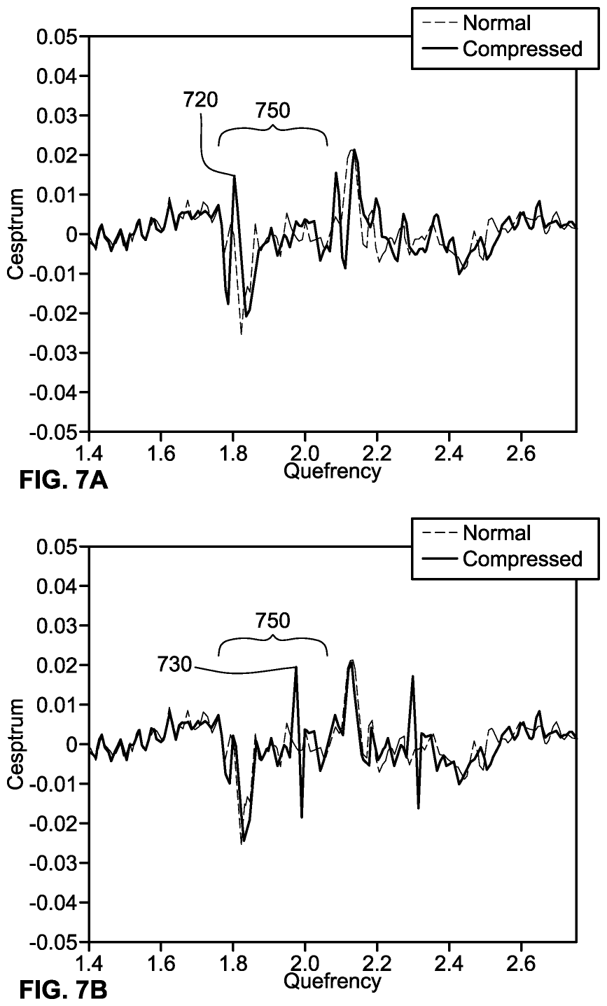

FIGS. 7A and 7B are plots of exemplary calculated cepstrums of generated acoustic data for a headgear user interface. The calculated cepstrums are an example of acoustic signatures for a condition of the headgear conduit. The dashed lines in FIGS. 7A and 7B represent a baseline acoustic signature for a non-occluded (“normal”) headgear conduit. The solid line in FIG. 7A represents a fully occluded (“compressed”) headgear conduit for a temple occlusion, such as temple occlusion 435a in FIG. 6. The temple occlusion is identifiable by the peak 720 that differs significantly in amplitude from the baseline signature at a quefrency of 1.8 and because the peak 720 lies within zone 750 that windows an acoustic reflection region of interest, such as the region 450a in FIG. 6. The solid line in FIG. 7B represents a fully occluded headgear conduit for a cheek occlusion, such as cheek occlusion 435b in FIG. 6. The cheek occlusion is identifiable by the peak 730 that varies significantly in amplitude from the baseline signature at a quefrency of 2.0 and because the peak 730 lies within zone 750 that windows the acoustic reflection region of interest, such as the region 450b in FIG. 6. It will be further understood that the region of interest (or window), e.g., region of the headgear conduit adjacent to the user's temple or cheek, may be determined simply by knowing the length of the conduit (tube) connecting the user interface to the respiratory therapy device. For example, the conduit for which the data in FIG. 6 are generated is approximately 1.8 m. In cases in which the conduit length is not known a priori, this can be estimated by finding the location of the user interface signature, i.e., characteristic pattern of features, in the cepstrum along the x-axis using any variety of methods known in the art.

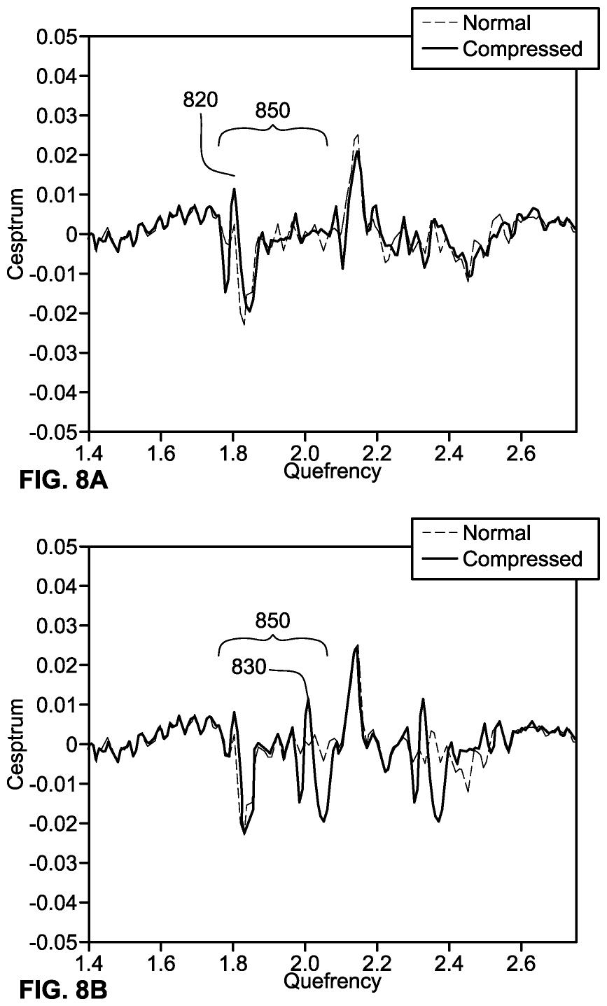

FIGS. 8A and 8B are also plots of exemplary calculated cepstrums of generated acoustic data for a different headgear user interface. The dashed lines in FIGS. 8A and 8B also represent a baseline acoustic signature for the non-occluded (“normal”) headgear conduit. The solid line in FIG. 8A represents a fully occluded (“compressed”) headgear conduit, also for a temple occlusion, such as temple occlusion 435a in FIG. 6. The temple occlusion is identifiable by the peak 820 that differs in amplitude from the baseline signature at a quefrency of 1.8 and because the peak 820 lies within zone 850 that windows an acoustic reflection region of interest, such as the region 450a in FIG. 6. The solid line in FIG. 8B represents a fully occluded headgear conduit for a cheek occlusion, such as cheek occlusion 435b in FIG. 6. The cheek occlusion is identifiable by the peak 830 that varies in amplitude from the baseline signature at a quefrency of 2.0 and because the peak 830 also lies within zone 850 that windows the acoustic reflection region of interest, such as the region 450b in FIG. 6.

The plots in FIGS. 7A to 8B are shown with quefrency in the horizontal x-axis and amplitude in the vertical y-axis. The quefrency is similar to time or distance, but the units of the horizontal x-axis are simply samples or sample numbers of the acoustic reflection that have been modified to approximately correspond with a distance along the air flow pathway from a microphone generating the acoustic data to an area where an acoustic signal was reflected. This allows for the approximation of a zone of interest (e.g., zones 750, 850) within the plot to identify amplitude differences.

The units of the vertical y-axis can be any unit that measures the amplitude. The vertical y-axis in FIGS. 7A to 8B are labeled as cepstrum. In some implementations, the y-axis cepstrum values can be understood to be an inverse Fourier transform of the logarithm of Fourier transform of the x-axis values. Amplitude differences between the baseline acoustic signatures and a current acoustic signature will increase as the degree of occlusion in a headgear conduit increases, thereby allowing a degree of occlusion to be estimated. It is contemplated that the degree of occlusion for a headgear conduit may be determined through the collection of baseline acoustic data for headgear conduits with known levels of occlusion to allow a percentage of occlusion or an occlusion value indicative of degree of occlusion to be determined. The calculation of the cepstrum can also be a function of the tube length, temperature, and humidity as each of these parameters affects the determination of an acoustic signature for comparison with baseline acoustic signatures or other types of analysis of the acoustic signature.

For the cepstrum plots of FIGS. 7A to 8B, a negative amplitude or trough in the acoustic signature plot can be indicative of a widening of a conduit cross-section and a positive peak can be indicative of a narrowing of a headgear conduit cross-section. In general, an acoustic reflection will occur any time there is a change in cross-sectional area, as this leads to a change in impedance. While there may be other second order effects due to the absolute cross sectional area involved, a relative change in conduit cross-section compared to a preceding section of the conduit will result in a negative or positive amplitude or trough.

The respiratory therapy system 120 generates acoustic data associated with an acoustic reflection of an acoustic signal. The acoustic data can be generated based on an acoustic transducer detecting the acoustic reflection. For example, the acoustic transducer can be a microphone that converts the pressure waves of the acoustic reflection into electrical signals representing the data. The acoustic reflection is indicative of, at least in part, one or more features of a user interface 124 coupled to a respiratory therapy device 122 via a tube 126. As the acoustic signal travels down the tube 126, the acoustic signal interacts with one or more features of the tube, one or more features of the connection between the tube and the user interface, one or more features of the user interface, etc. The interactions can cause the acoustic signal to reflect, thereby generating the acoustic reflection. In the context of an occlusion in a headgear conduit, because the acoustic reflection would be caused by such an occlusion in the user interface, the acoustic reflection is indicative of the occlusion.

In some implementations, the acoustic signal can be emitted into the tube connected to the user interface via an audio transducer, such as a speaker. Alternatively, or in addition, the acoustic signal can be emitted into the tube via a motor of the respiratory therapy device connected to the tube. For example, the motor of the respiratory therapy device can emit sound from the rotation of its fan blades as it generates air flow and forces air through the tube. The emitted sound can be the acoustic signal, or at least part of the acoustic signal.

In some implementations, the acoustic signal can be a sound audible to a human (e.g., an average human with average human hearing). A sound is audible to a human when the sound has an amplitude that is loud enough for detection by the human and when it has a frequency within the frequency range of human hearing, which is generally about 20 Hz to about 20,000 Hz. In some implementations, the acoustic signal can be a sound that is inaudible to a human (e.g., an average human with average human hearing). A sound may be inaudible when the sound has an amplitude that is less than the lowest perceptible amplitude of a human. In other examples, the acoustic signal can be an ultrasonic sound that has a frequency that is higher than the highest perceptible frequency for human hearing.

In one or more implementations, the generated acoustic data can be generated from a plurality of acoustic reflections of a plurality of acoustic signals. The acoustic signals can be the same acoustic signal that is repeatedly emitted, or can be different acoustic signals that are emitted together, forming a single acoustic signal, or can be emitted separately. When there are multiple acoustic signals for multiple acoustic reflections, the generated acoustic data can be an average of the plurality of acoustic reflections of the plurality of the acoustic signals.

In some implementations, the generated acoustic data can include a primary reflection within the acoustic reflection of the acoustic signal. Additionally, or in the alternative, the generated acoustic data can include a secondary reflection within the acoustic reflection of the acoustic signal. Additionally, or in the alternative, the generated acoustic data can include a tertiary reflection within the acoustic reflection of the acoustic signal. The generated acoustic data can be analyzed from the primary, secondary, or tertiary reflections. For example, the acoustic signatures used to detect or identify a conduit occlusion can relate to the primary reflection within the generated acoustic data. Alternatively, the acoustic signatures used to detect or identify a conduit occlusion can relate to a reflection other than the primary reflection within the generated acoustic data, such as a secondary reflection, a tertiary reflection, etc. Alternatively, the acoustic signatures used to detect or identify a conduit occlusion can include combinations of reflections, such as the first and second reflections; the first and third reflections; the first, second and third reflections; the second and third reflections, etc.

In some implementations, the magnitudes of a cepstrum corresponding to a primary reflection may be larger than the magnitudes of a cepstrum corresponding to a secondary reflection. However, the shapes of the plots for the primary and secondary reflections may be similar. In one or more implementations, acoustic signatures in a secondary reflection can be used to confirm acoustic signatures in a primary reflection with respect to detecting a conduit occlusion of the user interface. In one or more implementations, the presence/absence of additional reflections (e.g., a secondary reflection and/or tertiary reflection) can be used as an acoustic signature for detecting a conduit occlusion of the user interface. In one or more implementations, the ratio of the secondary reflection (and/or tertiary reflection) with respect to the primary reflection can be used as an acoustic signature for detecting an occlusion of the user interface.

The respiratory therapy system 120 analyzes the generated acoustic data to correlate the occlusion or non-occlusion features of the headgear conduit to one or more signatures within the generated acoustic data. The one or more signatures can include, for example, a maximum magnitude of a transform, a minimum magnitude of a transform, a standard deviation of a transform, a skewness of a transform, a kurtosis of a transform, a median of an absolute value of a transform, a sum of the absolute value of a transform, a sum of a positive area of a transform, a sum of a negative area of a transform, a fundamental frequency, an energy corresponding to the fundamental frequency, a mean energy, at least one resonant frequency of a combination of the conduit and the user interface, a change in the at least one resonant frequency, a number of peaks within a range, a peak prominence, distance between peaks, or a combination thereof. In one or more implementations, one or more of the signatures can be changes in one or more of the above aspects over time. The changes can be during a series of iterations of comparing determined acoustic signatures with baseline acoustic signatures, such as consecutive iterations over the course of a predetermined time period (e.g., ten seconds, twenty seconds, thirty seconds, one minute, a few minutes).

In some implementations, analyzing the generated acoustic data includes generating a spectrum of the generated acoustic data, such as calculating a transform (e.g., Discrete Fourier Transform DFT, or Discrete Cosine Transform DCT, Wavelet Transform (which also generates a “spectrum” like a frequency domain signal), Z/Laplace Transforms (which converts signals into other “domains”), etc.) of the generated acoustic data. Thereafter, analyzing the generated acoustic data can further include calculating a logarithm of the spectrum. Thereafter, analyzing the generated acoustic data can include calculating a cepstrum of the logarithm spectrum, such as calculating an inverse transform (e.g., iDFT or iDCT) of the logarithm spectrum. The calculated cepstrum can then be analyzed for one or more acoustic signatures that can be used to identify anomalies in a headgear conduit and determine if the identified anomaly relates to an occlusion.

In some implementations, the analysis of the generated acoustic data does not require analyzing all of the generated acoustic data. For example, analyzing the generated acoustic data can include selecting a segment of the above-described spectrum. After selecting the segment, the analysis can further include calculating a direct transform (e.g., DFT or DCT) of the segment of the spectrum. From the direct transform, the one or more features of the user interface can be correlated to one or more signatures within the Fourier transform of the segment to identify the category of the user interface. Alternatively, analyzing the generated acoustic data can include selecting a segment of the log spectrum of the generated acoustic data described above. From the log spectrum, the analysis can include calculating a transform of the log spectrum, such as a Fourier transform of the log spectrum. Alternatively, the analysis can include calculating an inverse transform of the log spectrum, or an inverse transform of the transform of the log spectrum.

In some implementations, a segment of the generated acoustic signal can be selected for analysis by trimming the generated acoustic data based on where within the generated acoustic data the one or more signatures that indicate where occlusions in the headgear user interface are generally located. This is generally based on a known location of the microphone and a known length of tube 126, at the end of which the headgear user interface and headgear conduits are located.

In some implementations, the acoustic signatures used to identify an occlusion in a headgear user interface can relate to the primary reflection within the generated acoustic data. Alternatively, the signatures used to categorize the user interface can relate to a reflection other than the primary reflection within the generated acoustic data, such as a secondary reflection, a tertiary reflection, etc. Alternatively, the signatures used to categorize the user interface can include combinations of reflections, such as the first and second reflections; the first and third reflections; the first, second and third reflections; the second and third reflections, etc.

According to some implementations, the determination of an occlusion in a headgear user interface can include the one or more signatures of the cepstrum, e.g., one or more peaks or troughs in the cepstrum, having a maximum magnitude larger than a threshold. For example, the threshold can be the value 0.1 in the vertical y-axis (see FIGS. 7A to 8B).

In some implementations, calculating the cepstrum can be achieved by processing the acoustic reflection in a series of stages: calculate the frequency spectrum of the acoustic reflection via a fast Fourier transform; take the natural logarithm of the absolute magnitude of this frequency spectrum; and perform the inverse Fourier transform and calculate the real part of the signal to produce a cepstrum. This process may be repeated over multiple time intervals and an average cepstrum may be estimated. The resulting cepstrum signal transforms the acoustic reflection into the quefrency domain which is analogous to separating reflections in time and/or distance. In one or more implementations, the cepstrum can be calculated using a fast Fourier transform of 4096 samples of the acoustic reflection with, optionally, 50% overlap of the samples. These 4096 samples represent about 0.2 seconds of generated acoustic data.

In some implementations, the analyzing of the generated acoustic data can include calculating a derivative of the cepstrum to determine a rate of change of the cepstrum signal. The derivative of the cepstrum can provide additional information that is determine if an identified anomaly relates to an occlusion.

In some implementations, the respiratory therapy system 120 analyzing the generated acoustic data can include normalizing the generated acoustic data. Normalizing the generated acoustic data can account for confounding conditions. The confounding conditions can be, for example, microphone gain, breathing amplitude, therapy pressure, varying tube length, curled or stretched tube, different therapy pressures, ambient temperature, or a combination thereof.

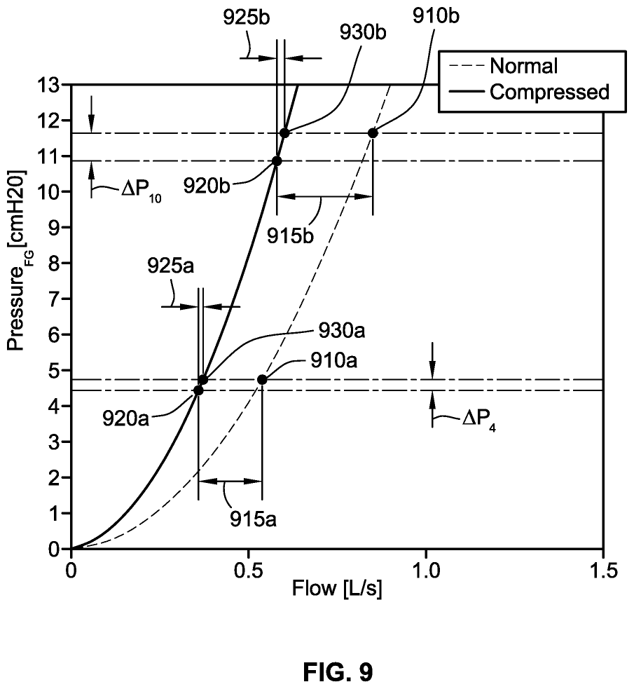

Turning now to FIG. 9, a plot of pressure-flow diagrams is depicted for a headgear user interface for a respiratory therapy system, where the solid curved line represents a flow-pressure curve for a headgear user interface with an occluded (“compressed”) headgear conduit, and the dashed curved line represents a flow-curve for the headgear user interface with non-occluded (“normal”) headgear conduits. The pressure-flow plots show the relationship between pressure and flow measured at the flow generator motor of a respiratory therapy device 122. The relationship is characterized by the combined impedance of the tube 126 and the user interface 124. The dashed curve plot on the right shows the required flow from the flow generator to achieve a certain pressure. For example, with an air flow just above 0.5 liters/second a pressure of about 4.8 cm H₂O is achieved at the motor for the respiratory therapy device 122 at indicated at point 910a. Similarly, with an air flow of about 0.9 liters/second a pressure of about 11.7 cm H₂O is achieved at the motor for the respiratory therapy device 122 at indicated at point 910b. With regard to the latter, this air flow and pressure at the motor can result in a pressure of about 10 cm H₂O at the user interface.

The solid curve plot shows the changes in pressure at the flow generator due to an increase in impedance in the headgear user interface as a result of a compressing of or occlusion in the headgear conduit of the user interface. For example, the occlusion causes a decrease in flow shown by element 915a of about 0.15 liters/second and a slight decrease in pressure, delta P, of about 0.2 cm H₂O at point 920a relative to the flow and pressure values at point 910a. Similarly, the occlusion causes a decrease in flow shown by element 915b of about 0.25 liters/second and a decrease in pressure, delta P, of about 1.0 cm H₂O at point 920b relative to the flow and pressure values at point 910b.

The plots are particularly beneficial because they provide a useful approximation of the amount of increased flow that is needed at the motor of the respiratory therapy device 122 to achieve the same pressurized air flow as before the occlusion was identified. For example, an anomaly may be identified from determined acoustic signatures that relate to an occlusion in a headgear conduit. The occlusion can further be verified by a decrease in flow in the respiratory therapy system. In response, the motor of the respiratory therapy device can compensate for a pressure drop by modifying air flow settings during a therapy session by increasing air flow to the value at point 930b if the non-occluded pressure was the value for point 910b. Similarly, in the example of the non-occluded condition having a normal air flow pressure that was the value for point 910a, the motor of the respiratory therapy device can compensate for a pressure drop by modifying air flow settings during a therapy session by increasing air flow to the value for point 925a. The increase in air flow only approximates a compensation for the pressure drop. To achieve a more complete compensation of the pressure drop at the mask due to the occlusion, a correction can also be applied to factor in that the tube impedance also changes with flow. To calculate the correction, the pressure drop for the tube is recalculated using the newly measured decreased flow with the occlusion.

The plot in FIG. 9 can be generated for other respiratory therapy system and air flow pathway configurations for a headgear user interface to approximate the air flow modifications needed to achieve a desired therapy pressure at the headgear user interface. In some implementations, the slope of the normal and compressed pressure-flow curved can be approximated to be the same where the slope from the normal pressure-flow curve can be used to estimate the approximate increase in flow needed to achieve the desired air flow pressure.

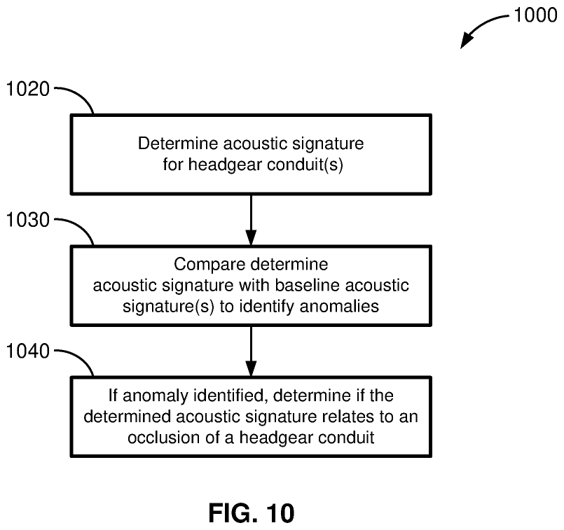

FIG. 10 is a flow diagram of a process 1000 for determining if a headgear user interface has an occlusion. The process can be implemented on one of more of the systems of system 100 described for FIG. 1. Starting at step 1020, an acoustic signature for the conduit of the headgear user interface is determined. Then, at step 1030, the determined acoustic signature is compared with one or more baseline acoustic signatures associated with the headgear user interface to identify an anomaly. Alternatively, at step 1030, the determined acoustic signature is analyzed to identify an anomaly or anomalies in the determined acoustic signature(s). Next, at step 1040, a determination is made if the identified anomaly relates to an occlusion of the conduit of the headgear user interface if the comparison or analysis in step 1030 suggests an anomaly.

In some implementations, an initial identification can be made before step 1120, that a headgear user interface having a headgear conduit is being used. This step can be completed based on user inputs or acoustic techniques as disclosed, for example, in U.S. Application No. 63/036,303, filed Jun. 8, 2020, entitled, “Systems and Methods for Characterizing a User Interface”, U.S. Application No. 63/108,161, filed Oct. 30, 2020, entitled, “Systems and Methods for Categorizing and/or Characterizing a User Interface”, and International (PCT) Application No. PCT/IB2021/054999, filed Jun. 7, 2021, entitled, “Systems and Methods for Categorizing and/or Characterizing a User Interface” published on Dec. 16, 2021 as International Publication No. WO 2021/250553, the disclosures of which are hereby incorporated by reference herein in their entireties.

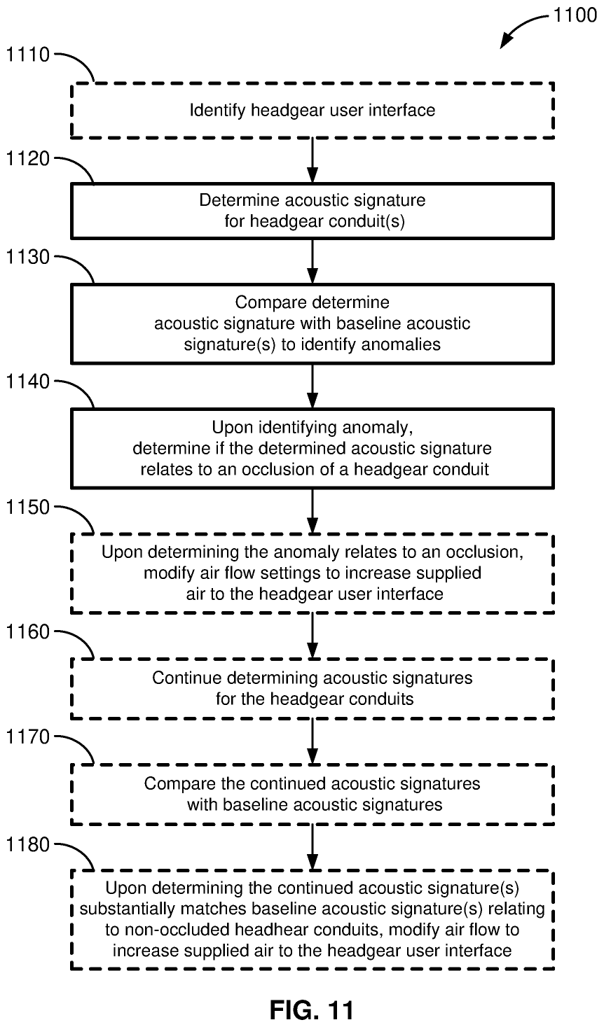

FIG. 11 is a flow diagram of a process 1100 for determining if a headgear user interface has an occlusion, and optionally modifying air flow (and/or determining a modification to the air flow) and continuing to determine if the occlusion persists. At step 1120, an acoustic signature is determined for the one or more headgear conduits during operation of the respiratory therapy system. Then, at step 1130, the determined acoustic signature is analyzed to identify an anomaly or anomalies in the determined acoustic signature(s). In some implementations, at step 1130, a comparison is alternatively made of the determined acoustic signature with one or more baseline acoustic signatures associated with the headgear user interface. The comparison identifies anomalies during operation of the respiratory therapy system. Next, at step 1140, in response to the determined acoustic signature being identified as anomalous, a determination is made if an identified anomaly in the determined acoustic signature relates to an occlusion of at least one of the one or more headgear conduits.

In some implementations, an optional preliminary step 1110 may be performed where a headgear user interface including one or more headgear conduits is identified. This step can be completed based on user inputs or acoustic techniques similar to those discussed for FIG. 10 at step 1010. Next, a

In some implementations, an optional step 1150 includes modifying air flow settings (and/or determining a modification to air flow settings) in the respiratory therapy system during a therapy session to increase air flow to the headgear user interface in response to determining the identified anomaly from the determined acoustic signature relates to an occlusion. In another optional step 1160, acoustic signatures are continuously determined for the one or more headgear conduits during a therapy session using the respiratory therapy system in response to modifying therapy flow setting in step 1150. In another optional step 1170, at least one of the continued determinations of acoustic signatures is compared with one or more baseline acoustic signatures associated with the one or more headgear conduits during a therapy session using the respiratory therapy system. Then, in another optional step 1180, in response to at least one of the continued determinations of acoustic signatures substantially matching one or more baseline acoustic signatures relating to non-occluded headgear conduits, air flow in the respiratory therapy system is modified during a therapy session to achieve desired headgear user interface pressure settings for the respiratory therapy system.

While the two processes 1000, 1100 have been shown and described herein as occurring in a certain order, more generally, the steps of either of process can be performed in any suitable order.

One or more elements or aspects or steps, or any portion(s) thereof, from one or more of any of claims 1 to 72 below can be combined with one or more elements or aspects or steps, or any portion(s) thereof, from one or more of any of the other claims 1 to 72 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for detecting an occlusion within a respiratory therapy system, the method comprising:
determining an acoustic signature for one or more headgear conduits included in a headgear user interface comprising a plurality of headgear conduits during operation of the respiratory therapy system;
analyzing the determined acoustic signature to identify anomalies in the acoustic signature during operation of the respiratory therapy system; and
in response to the determined acoustic signature being identified as anomalous, determining if an identified anomaly in the determined acoustic signature relates to an occlusion of at least one of the one or more headgear conduits.

2. The method of claim 1, further comprising identifying the headgear user interface.

3. The method of claim 1, wherein the analyzing of the determined acoustic signature includes comparing the determined acoustic signature with one or more baseline acoustic signatures associated with the headgear user interface to identify anomalies in the acoustic signature.

4. The method of claim 1, further comprising in response to determining the identified anomaly from the determined acoustic signature relates to an occlusion, determining a modification to air flow settings in the respiratory therapy system during a therapy session to increase air flow to the headgear user interface.

5. The method of claim 4, wherein the modification of the therapy flow settings includes increasing air flow to achieve a desired headgear user interface pressure setting for the therapy session.

6. The method of claim 4, wherein the increasing of air flow to achieve a desired headgear user interface pressure setting includes increasing air flow, via a flow generator motor of the respiratory therapy system, based on predetermined pressure-flow relationships for (i) a headgear conduit having an occlusion, (ii) a headgear conduit that is unobstructed, or both (i) and (ii).

7. The method of claim 4, wherein the increased air flow for an occluded headgear conduit is less than the air flow for a non-occluded headgear conduit during the therapy session.

8. The method of claim 1, further comprising in response to determining the identified anomaly from the determined acoustic signature relates to an occlusion, (i) storing information related to a detected occlusion in the one or more headgear conduits for analysis by a body position algorithm module and/or a body movement algorithm module associated with the respiratory therapy system, and/or (ii) inputting information related to a detected occlusion in the one or more headgear conduits into a body position algorithm module and/or a body movement algorithm module associated with the respiratory therapy system.

9. The method of claim 1, wherein the determining if an identified anomaly in the determined acoustic signature relates to an occlusion includes the identified anomaly being continuous for a series of acoustic signature determinations over a predetermined period.

10-12. (canceled)

13. The method of claim 4, further comprising:
in response to an implemented modification to therapy flow settings after determining the identified anomaly in the determined acoustic signature relates to an occlusion, continuing to determine acoustic signatures for the one or more headgear conduits during the therapy session using the respiratory therapy system;
comparing at least one of the continued determined acoustic signatures with one or more baseline acoustic signatures associated with the one or more headgear conduits during a therapy session using the respiratory therapy system;
in response to at least one of the continued determined acoustic signatures substantially matching one or more baseline acoustic signatures relating to non-occluded headgear conduits, determining a further modification to air flow in the respiratory therapy system during a therapy session to achieve desired headgear user interface pressure settings for the respiratory therapy system.

14-15. (canceled)

16. The method of claim 1, wherein a total length of at least one of the one or more headgear conduits extends from a connector at one end of the one or more headgear conduits to a second end at a cushion or a frame of the headgear user interface, the connector coupling the tube conveying air from a respiratory therapy device of the respiratory therapy system to the one or more headgear conduits, the determined acoustic signature and the one or more baseline acoustic signatures being associated with at least a predefined region of the one or more headgear conduits, the predefined region being less than the total length.

17. The method of claim 1, wherein the determined acoustic signature is based on acoustic data received from a microphone associated with the respiratory therapy system, wherein the microphone is coupled to a component of the respiratory therapy system along an air pathway formed by a respiratory therapy device, tube, and the headgear user interface of the respiratory therapy system.

18-22. (canceled)

23. The method of claim 1, wherein the identified anomaly in the determined acoustic signature relates to one of (i) a partial occlusion of one of the one or more headgear conduits or (ii) a substantially full occlusion of one of the one or more headgear conduits.

24. (canceled)

25. The method of claim 1, wherein the determining if an identified anomaly in the determined acoustic signature relates to an occlusion includes comparing the determined acoustic signature with a plurality of baseline acoustic signatures associated with partial to full occlusions of headgear conduits of the headgear user interface.

26. The method of claim 25, further including determining a degree of occlusion by comparing the determined acoustic signature with the plurality of baseline acoustic signatures associated with the partial to full occlusions of headgear conduits of the headgear user interface.

27-30. (canceled)

31. The method of claim 1, wherein the determined acoustic signature is based on calculating a cepstrum of the acoustic data, wherein the cepstrum identifies a distance associated with the one or more acoustic reflections, the distance indicating a location of the identified anomaly.

32-36. (canceled)

37. A method for detecting an occlusion in a respiratory therapy system, the method comprising:
determining an acoustic signature for a conduit of included in a headgear user interface comprising a plurality of headgear conduits;
analyzing the determined acoustic signature to identify an anomaly in the acoustic signature; and
in response to the determined acoustic signature being identified as anomalous, determining if the identified anomaly relates to an occlusion of the conduit.

38-72. (canceled)

73. A system for detecting an occlusion within a respiratory therapy system, the system comprising:
a memory having stored thereon machine readable instructions;
a control system including one or more processors configured to execute the machine-readable instructions to:
determine an acoustic signature for one or more headgear conduits included in a headgear user interface comprising a plurality of headgear conduits during operation of the respiratory therapy system;
analyze the determined acoustic signature to identify anomalies in the acoustic signature during operation of the respiratory therapy system; and
in response to the determined acoustic signature being identified as anomalous, determine if an identified anomaly in the determined acoustic signature relates to an occlusion of at least one of the one or more headgear conduits.

74. The system of claim 73, wherein the control system is further configured to execute the machine-readable instructions to identify the headgear user interface.

75. The system of claim 73, wherein the control system is further configured to execute the machine-readable instructions to:
in response to determining the identified anomaly from the determined acoustic signature relates to an occlusion, determining a modification to air flow settings in the respiratory therapy system during a therapy session to increase air flow to the headgear user interface.
